(19) United States
(12) Patent Application Publication
(10) Pub. No.: US 2023/0235261 A1
(43) Pub. Date: Jul. 27, 2023

(54) DROPLET TRAPPING STRUCTURE ARRAY, METHOD FOR SPHEROID TRANSFER AND FORMATION OF SPHEROID ARRAY USING THE SAME

(71) Applicant: KOREA ADVANCED INSTITUTE OF SCIENCE AND TECHNOLOGY, Daejeon (KR)

(72) Inventors: Je-Kyun PARK, Daejeon (KR); Hwisoo KIM, Chungcheongbuk-do (KR)

(21) Appl. No.: 18/157,834

(22) Filed: Jan. 22, 2023

(30) Foreign Application Priority Data
Jan. 25, 2022 (KR) 10-2022-0010679

(51) Int. Cl. C12M 1/12; C12M 1/32; C12N 5/00

(52) U.S. Cl. CPC C12M 25/01; C12M 23/12; C12N 5/0062; C12N 2513/00

(57) ABSTRACT

Disclosed are a spheroid array and more particularly, a droplet trapping structure array capable of isolating all or selected spheroids into an isolated droplet array environment and the use thereof. The droplet-trapping structure array and the method and device for transferring spheroids using the same have the advantages of transferring droplets or spheroids with very high efficiency and very small variation between users by simply contacting two arrays. The spheroid transfer method and device enable mass-production of spheroid arrays in an isolated environment. In particular, the droplet trapping structure array and the spheroid transfer method can be useful for the treatment of spheroids with various reagents and the exchange of culture media.

### CROSS-REFERENCE TO RELATED APPLICATION

The priority under 35 USC § 119 of Korean Patent Application 10-2022-0010679 filed Jan. 25, 2022 is hereby claimed, and the disclosure thereof is hereby incorporated herein by reference, in its entirety, for all purposes.

### BACKGROUND OF THE INVENTION

### Field of the Invention

The present invention relates to a spheroid array, particularly a droplet trapping structure array capable of isolating all or selected spheroids into an isolated droplet array environment and the use thereof.

### Description of the Related Art

Spheroids are spherical cell aggregates and are useful 3D cell culture models for a wide range of applications due to their simple manufacturing method (E. Fennema et al., Trends Biotechnol., 2013, 31, 108-115). Methods for producing spheroids include a method of producing a single spheroid for each compartment, such as a hanging drop or low-attachment well plate, and a method of producing large-scale spheroids in a single medium such as a spinner flask, a microwell array, and a hanging drop microarray presented in Patent Document 1.

However, conventional methods have the following limitations in applying spheroids to high-efficiency drug treatment experiments. First, the hanging drop and the low-attachment well plate require a number of pipette operations corresponding to the desired number of spheroids, since they are compartmentalized and spheroids are not simultaneously produced by a single pipetting. This can be solved with a multi-channel pipette or a pipetting machine capable of simultaneously pipetting in multiple compartments, but it has the drawbacks of lower generation efficiency and the necessity of expensive and bulky equipment compared to the subsequent large-scale spheroid production methods. The spinner flask produces spheroids on a large scale but with poor homogeneity, whereas microwell arrays and hanging drop microarrays can produce uniform spheroids on a large scale. However, a potential issue with these methods is that the spheroids are connected to each other through large liquid reservoirs, allowing for interaction between them spheroids. The hanging drop microarray is capable of simultaneously retrieving spheroids in the spheroid production device through contact with other liquids contained in the well, but it is also unsuitable for multi-condition treatment because it moves to the same well.

Meanwhile, R. Tomasi et al. has proposed a microfluidic platform to generate spheroid droplet arrays and to perform multiplexed drug treatment (R. Tomasi et al., Cell Reports., 2020, 31, 107670). In this study, spheroids were produced by rapidly generating cell suspension droplets in oil, taking advantage of the immiscibility between oil and water. The generated spheroid droplets were then immobilized in specific regions of the microfluidic platform by utilizing changes in the height difference within the microfluidic channel. In addition, various drug droplets were created using different drugs, and the drug droplets were fixed around the spheroid droplets and merged together by using a microstructure that was designed to consider droplet movement caused by interfacial tension. At this time, spheroid droplets remain compartmentalized from each other due to the oil surrounding each droplet. However, in this study, the injection of droplets is always based on driving using a syringe pump and thus it is difficult to control the number or position of spheroids to be reacted. In addition, as the spheroid droplets are fixed in the channel of the device, it is also difficult to collect specific spheroids. Therefore, a method for forming compartmentalized spheroid arrays at high efficiency that has excellent spheroid accessibility and can be easily operated is still challenging.

Accordingly, the present inventors have developed and reported a technique called droplet contact-based spheroid transfer (DCST), which is capable of simultaneously transferring and culturing spheroid arrays in a compartmentalized collagen hydrogel array (H. Kim et al., Biomicrofluidics, 2018, 12, 044109). In the document, the present inventors found that spheroids can be transferred to other media or hydrogels through simple droplet contact while minimizing the risk of spheroid loss and contamination due to direct pipetting. They achieved this by moving the spheroids in such a way that a pillar array chip (PAC), including pillars capable of capturing spheroids that are repeatedly arranged, is brought into contact with a drop array chip (DAC). However, the pillars arranged in the pillar array chip of the document have a flat top surface and thus have a problem that the droplets flow out of the pillars and the spheroids are lost during long-term culture or repeated droplet contacts.

Under this background art, as a result of diligent and extensive efforts to solve the above problems, the present inventors have developed a contact-based droplet trapping structure array, including a concave plateau in contact with droplets. When using this structure for droplet contact-based spheroid transfer, it exhibits a significantly higher spheroid transfer rate than the previously reported flat-top pillar array chip, even during repeated droplet contact. In particular, it exhibits a higher spheroid transfer rate even for larger volume droplets when air channels are formed in the concave plateau. The use of not only pillars but also various types of droplet trapping arrays also results in a high spheroid transfer rate. The present inventors have confirmed that this technology can be used to easily compartmentalize large-scale spheroids into separated spheroid droplets. Furthermore, the present inventors found that excellent efficiency and effects can be obtained regardless of the skill of the operator when using the droplet contact-based spheroid transfer (DCST) for various applications such as reagent treatment and culture medium exchange. Based thereon, the present invention has been completed.

### SUMMARY OF THE INVENTION

It is one object of the present invention to provide a contact-based droplet trapping structure array capable of trapping droplets at a high transfer rate from the drop array.

It is another object of the present invention to provide a method and device for transferring droplets or spheroids, including the contact-based droplet trapping structure array.

It is another object of the present invention to provide a method for treating spheroids with a reagent or exchanging a medium using the contact-based droplet trapping structure array.

In accordance with one aspect of the present invention, the above and other objects can be accomplished by the provision of a contact-based droplet trapping structure array, including a concave plateau.

In accordance with another aspect of the present invention, provided is a droplet transfer device or droplet trapping device including a drop array and the contact-based droplet trapping structure array.

In accordance with another aspect of the present invention, provided is a spheroid transfer device including a spheroid array and the contact-based droplet trapping structure array.

In accordance with another aspect of the present invention, provided is a droplet transfer method including (a) bringing a drop array into contact with the contact-based droplet trapping structure array of the present invention, and (b) releasing the contact between the drop array and the contact-based droplet trapping structure array to trap droplets in a concave plateau of the contact-based droplet trapping structure array.

In accordance with another aspect of the present invention, provided is a droplet contact-based spheroid transfer method including (a) bringing a spheroid array into contact with the contact-based droplet trapping structure array of the present invention, and (b) releasing the contact between the spheroid array and the contact-based droplet trapping structure array to transfer droplets containing spheroids to the concave plateau of the contact-based droplet trapping structure array.

In accordance with another aspect of the present invention, provided is a method for producing a spheroid array using the spheroid transfer method.

In accordance with another aspect of the present invention, provided is an isolated spheroid array produced by the method of producing the spheroid array.

In accordance with another aspect of the present invention, provided is a method of treating spheroids with a reagent, including (a) bringing a cultured spheroid array into contact with the contact-based droplet trapping structure array according to the present invention, (b) releasing the contact between the cultured spheroid array and the contact-based droplet trapping structure array to produce an isolated spheroid droplet array, and (c) bringing the isolated spheroid droplet array into contact with a reagent-loaded drop array.

In accordance with another aspect of the present invention, provided is a method of exchanging a spheroid medium, including (a) bringing a cultured spheroid array into contact with the contact-based droplet trapping structure array according to the present invention, (b) releasing the contact between the cultured spheroid array and the contact-based droplet trapping structure array to produce an isolated spheroid droplet array, and (c) bringing the isolated spheroid droplet array into contact with a medium-loaded drop array to transfer spheroids.

### BRIEF DESCRIPTION OF THE DRAWINGS

The above and other objects, features, and other advantages of the present invention will be more clearly understood from the following detailed description taken in conjunction with the accompanying drawings in which:

FIG. 1 is a schematic diagram illustrating, in parts A, B, C, and D thereof, an assay platform for hanging drop spheroid arrays using a droplet contact-based spheroid transfer (DCST) device, in which: part A illustrates an operation mechanism of the assay platform using a DCST method, part B illustrates a design of the DCST device: a drop array chip (DAC), a pillar array chip (PAC) and a spacer, part C illustrates a process of manufacturing the DCST device, and part D illustrates a detailed process of each reagent change step;

FIG. 2 shows the effect of silanization on PDMS molding wherein images A and B show a PDMS-based pillar array chip made from uncoated PlasCLEAR mold (image A) and its magnified image (image B), and images C and D show a PDMS-based pillar array chip made from silanized PlasCLEAR mold (image C) and its magnified image (image D);

FIG. 3 in parts A-E thereof shows the characterization of the DCST-based multi-step assay platform, wherein: part A shows a pillar-shaped contact-based droplet trapping structure (pillar array chip; PAC), including a concave plateau and air channels, parts B and C show changes in the transferred drop volume (part B) and spheroid transfer rate (part C) depending on the pillar diameter of the PAC (spheroid transfer rate=the number of spheroids after transfer/the number of spheroids before transfer), parts D and E show the washing efficiency as a function of the number of contacts, wherein part D shows a schematic diagram (left) and an image (right) of the washing procedure using repeated DAC-PAC contacts, and part E illustrates the residual amount (upper part) and washing efficiency (lower part) of the first reagent compared to the number of contacts with the washing droplet and washing efficiency (%)=(1−current concentration of first reagent droplet/initial concentration of first reagent droplet)×100;

FIG. 4 shows the growth of BT-474 spheroids over 14 days in 384-well spheroid microplates and drop array chips using manual pipetting and droplet contact-based spheroid transfer (DCST), wherein the initial number of BT-474 cells constituting the spheroid is 4000 for both devices;

FIG. 5 in parts A-D thereof shows the result of the comparison in user deviation between the conventional manual pipetting method and the DCST method in the medium (or reagent) replacement process, more specifically, part A is a schematic diagram illustrating a medium (or reagent) replacement method using manual pipetting in a conventional microwell plate, part B is a schematic diagram illustrating a medium (or reagent) replacement method using the DCST method, and parts C and D show the results of the comparison in the spheroid retention rates between the two methods for seven individuals and the average retention rates of the three different methods, respectively, wherein one-way ANOVA with Games-Howell post hoc test was used (n.s.: not significant, p>0.05, *p<0.05, and **p<0.01);

FIG. 6 shows the spheroid retention rate using an automated pipetting platform and the calculated volume of residual liquid depending on the z-position of the pipette tip;

FIG. 7 in parts A-D thereof shows DCST-based chemical staining and immunostaining of BT-474 spheroids, more particularly, part A shows the live/dead and hypoxia staining results of BT-474 spheroids (scale bar=500 μm), parts B and C show the results of immunofluorescence staining of hypoxia-inducible factor-1 alpha (HIF-1α) (part B) and E-cadherin (part C) depending on primary antibody concentrations, respectively, (Scale bar=1 mm), and part D shows the result of comparison in the immunostaining uniformity between a conventional spheroid microplate using manual pipetting and a DCST device using the DCST method;

FIG. 8 is an image showing that spheroids are attached to a device that is not coated with BSA, wherein white dots indicated by red arrows are spheroids attached to the drop array chip (scale bar=5 mm);

FIG. 9 in parts A-C thereof shows the results of spheroid clearing using RTF, ScaleSQ, modified ScaleS, and FOCM clearing methods, including (part A) brightfield image, (part B) fluorescent image of the center of the spheroid, and (part C) fluorescence images at different z-axis heights and sides;

FIG. 10 shows, as a spheroid array used in the droplet contact-based spheroid transfer device prepared in Example of the present invention, a hanging drop microarray having a plurality of holes at the bottom thereof (International Patent Publication No. WO2015/129263);

FIG. 11 is an image showing the results of the generation/culture of spheroids over time using a hanging drop microarray having a plurality of holes at the bottom thereof and live/dead staining results;

FIG. 12 shows a concave pillar-type droplet contact-based trapping structure including an air channel produced according to an embodiment of the present invention and a microarray including the same;

FIG. 13 shows a droplet contact-based trapping structure with a concave plateau, including air channels and microwalls on a flat surface thereof;

FIG. 14 is a schematic diagram illustrating a method of transferring droplets containing spheroids to the contact-based droplet trapping structure array of the present invention using the droplet contact-based spheroid transfer device of the present invention and the results thereof, wherein in part A all spheroids of the spheroid array are transferred, and wherein in part B some spheroids of the spheroid array are selectively transferred;

FIG. 15 shows a method for transferring cultured spheroids to a collagen hydrogel drop array using the contact-based droplet trapping structure array of the present invention, and more particularly, part (i) therein shows a method for producing an isolated spheroid array by transferring cultured spheroids to the contact-based droplet trapping structure array of the present invention, part (ii) therein shows a method for producing a hydrogel-supported microarray (HSMA) by transferring a collagen hydrogel without spheroids to the contact-based droplet trapping structure array of the present invention, and part (iii) therein shows a method for finally transferring spheroids to a collagen hydrogel by bringing an isolated spheroid array into contact with a hydrogel supported microarray (HSMA), wherein the right portion of the figure shows the results of each step, and the red, yellow, and blue boxes represent steps (i), (ii), and (iii), respectively;

FIG. 16 shows the result of confirming the changes in spheroids and the size after culture when the spheroid-hydrogel droplet array is formed by transferring to a collagen hydrogel droplet array using the droplet trapping structure array device of the present invention; and

FIG. 17 shows the results of spheroid transfer performed using three designs (flat plateau, concave plateau, and concave plateau with air channels) of pillar array chip (PAC) with a BT-474 spheroid with a diameter of 450 μm and a top width of 1 mm.

### DETAILED DESCRIPTION OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

Spheroids are spherical cell aggregates formed by aggregating cells and are useful for a wide range applications due to simple three-dimensional culture manufacturing method thereof. However, previously reported methods for generating large-scale spheroids have a problem in that interactions between spheroids may occur and they are not isolated from each other. In order to test the spheroids under different conditions, the test is conventionally performed through repetitive operation by manual pipetting or using expensive automatic pipetting devices, but nonuniform results are obtained due to the cost, procedure, operator's skill, and the like.

In order to solve this problem, the present inventors developed a contact-based droplet trapping structure array including a concave plateau (H. Kim et al., Biomicrofluidics, 2018, 12, 044109) by improving the previously reported droplet contact-based spheroid transfer (DCST).

In one embodiment of the present invention, it was found that, with a contact-based droplet trapping structure array including a concave plateau, a mass-cultured spheroid array can be separated in the form of an isolated spheroid-droplet by a simple contact/release step. In particular, it was found that the contact-based droplet spheroid transfer using the droplet trapping structure array of the present invention reduces damage and loss of spheroids and variation between users compared to the conventional manual pipetting method.

In another embodiment of the present invention, the droplet contact-based spheroid transfer using the droplet trapping structure array of the present invention is useful for not only trapping droplets containing spheroids, but also exchange of medium, treatment of reagents, staining of spheroids, transparency and washing.

Accordingly, in one aspect, the present invention is directed to a contact-based droplet trapping structure array including a concave plateau.

The contact-based droplet trapping structure array of the present invention includes a concave plateau and thus has superior droplet transfer volume and spheroid transfer rate to a pillar array chip (PAC) having a flat-top surface previously reported by the present inventors (H. Kim et al., Biomicrofluidics, 2018, 12, 044109) and can prevent spheroids from being lost even during repeated droplet contact.

As used herein, the term “contact-based droplet trapping structure array” refers to a substrate on which a plurality of droplet contact-based trapping structures are arrayed and the array is used interchangeably with “array device” and “array chip” which have substantially the same meaning.

As used herein, the term “concave plateau” refers to a portion recessed from a horizontal surface. In the present invention, in a broad sense, any portion may be used as the concave plateau regardless of shape as long as it includes a recessed part from the horizontal plane. In the present invention, the concave plateau may mean that the concave plateau goes downward relative to a horizontal surface since the transfer of droplets or spheroids occurs due to gravity.

In one embodiment of the present invention, the droplet contact-based trapping structure is produced as a pillar array chip (PAC) by which it includes a pillar having a concave plateau on the top surface.

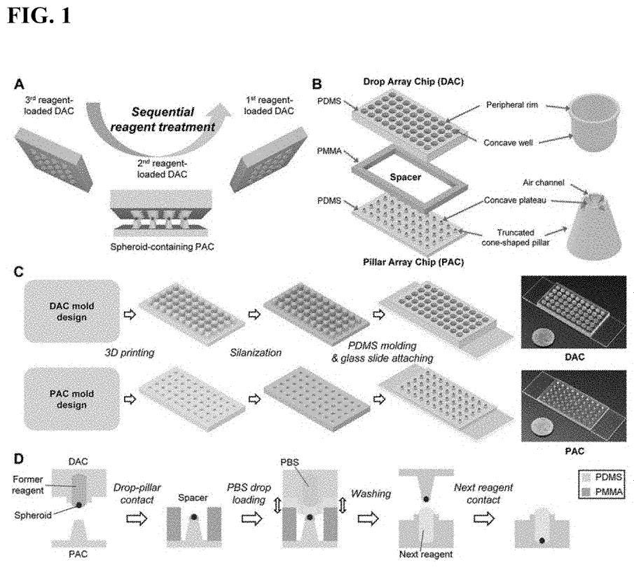

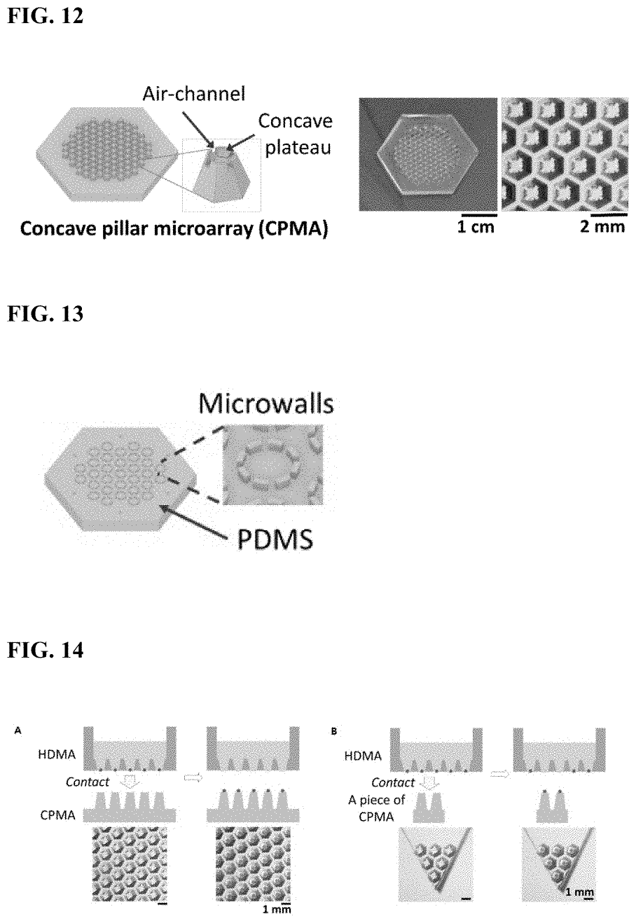

In the present invention, the concave plateau is formed on the top surface of the droplet contact-based trapping structure. In the present invention, the droplet contact-based trapping structure includes a polygonal pillar such as a triangular pillar or a square pillar, and includes a truncated horn-shaped pillar whose cross-sectional area narrows or widens toward the top. In one embodiment of the present invention, as shown in FIGS. 1 and 12, the pillar droplet contact-based trapping structure is produced in a form including a concave plateau at the top of a truncated cone or a truncated hexagon, but is not limited thereto.

In the present invention, the concave plateau of the pillar-type droplet contact-based trapping structure may be formed by cutting the center of the flat cross-section of the pillar, or may be formed by erecting microwalls on the flat cross section of the pillar of the concave plateau, but is not limited thereto.

In the present invention, the concave plateau may be formed on a flat plane. In one embodiment of the present invention, the pillar-type droplet contact-based trapping structure was initially designed to adjust the contact interval and provide easiness, but the liquid droplets could be effectively trapped from the drop array even when manufactured by forming microwalls on the flat plane to construct a concave plateau. As shown in FIG. 13, the concave structure may be formed by forming the microwalls in a circular shape, but is not limited thereto.

In the present invention, any portion may be used as the concave plateau regardless of the shape thereof as long as it has a concave structure compared to a planar cross section.

In the embodiment of the present invention, when the spheroid droplets are transferred through the contact-based droplet trapping structure array in the form of a concave pillar, air is trapped between the droplet and the concave plateau to form an air layer, resulting in a decrease in the volume of the transferred droplets as compared to the droplet trapping structure array with a flat top surface and loss of about 6-12% of spheroids. In order to overcome this problem, an air channel was formed in the concave plateau. As a result, stable droplet trap is possible due to the larger transferred droplet volume, and the spheroid transfer rate becomes 100%.

In the present invention, the concave plateau may include an air channel. In the present invention, the air channel may be formed on the side surface of the concave plateau. In the present invention, the concave plateau may include one or more air channels, preferably 2 to 6 air channels, and most preferably 4 or more air channels.

The contact-based droplet trapping structure array of the present invention can be used to form an isolated spheroid droplet array by trapping spheroid droplets from the spheroid array.

In the present invention, the droplet may contain a spheroid.

In the present invention, it is preferable that, when the contact-based droplet trapping structure array is used to transfer the spheroid, the inner shape of the concave plateau is hemispherical or semi-elliptical in order to dispose the spheroid in the central portion, since the spheroid has a spherical or elliptical spherical shape, but is not limited thereto. In the present invention, when the concave structure is formed with the microwall, the inner shape of the concave plateau may take the form of a cylindrical or polygonal pillar, and the inside of the microwall may be processed to be curved to form a hemispherical or elliptical shape.

In the present invention, the size of the concave plateau can be controlled depending on the type and volume of the droplet to be transferred. For example, when the concave plateau is used to transfer spheroids, it preferably has a diameter of 1 μm to 1 mm as prepared in Examples of the present invention, but is not limited thereto.

In one embodiment of the present invention, when the contact-based droplet trapping structure array including a concave plateau was used to transfer droplets containing spheroids, the spheroid transfer rate was determined depending on the spheroid size and the diameter of the concave plateau, and when the diameter of the spheroid was larger than that of the pillar, the spheroid transfer rate was greatly reduced.

In the present invention, when the contact-based droplet trapping structure array, including a concave plateau is used to transfer droplets containing spheroids, the width of the concave plateau may be larger than the diameter of the spheroids. The width of the concave plateau may be preferably at least 100 μm, more preferably at least 200 μm, and most preferably at least 300 μm larger than the diameter of the spheroids.

In one embodiment of the present invention, the concave plateau is produced to have a hemispherical or cylindrical shape having a depth of ½ of the width, but is not limited thereto.

In one embodiment of the present invention, the contact-based droplet trapping structure array is produced by curing PDMS in a 3D printed mold, but is not limited thereto and may be produced using various materials.

In the present invention, the contact-based droplet trapping structure array is characterized in that droplets are trapped in the concave plateau of each droplet contact-based trapping structure through contact with a drop array and release therefrom. The drop array vertically contacts with the concave plateau of the contact-based droplet trapping structure array of the present invention, and the droplets are transferred and trapped by gravity.

Therefore, in the present invention, the droplet contact-based trapping structure is preferably characterized in that the array that contacts the drop array is arranged in an identical or similar manner to the arrangement of the drop (droplet) array.

In the present invention, when some droplets of the drop array are selectively transferred using the contact-based droplet trapping structure array, the arrangement of droplet trapping structures may be different from that of the drop array and only some arrays may be arranged to contact the drop array.

In another aspect, the present invention is directed to a droplet transfer device or droplet trapping device, including a drop array and the contact-based droplet trapping structure array.

As used herein, the term “drop array” or “droplet array” refers to an array device in which liquid-loaded compartments are arranged. In one embodiment of the present invention, a drop array is formed by loading a liquid on a drop array chip conforming to the microwell plate standard and then flipping the chip, or a hanging drop microarray having a plurality of holes at the bottom is used, but is not limited thereto.

In the present invention, the droplet may contain a spheroid, and at this time, the spheroid is transferred to the concave plateau of the contact-based droplet trapping structure array by gravity along with the trap of the droplet. In the present invention, the drop array may be a spheroid array including droplets containing spheroids.

Therefore, in another aspect, the present invention is directed to a spheroid transfer device, including a spheroid array and the contact-based droplet trapping structure array.

As used herein, the term “spheroid” refers to a spherical cell aggregate (E. Fennema et al., Trends Biotechnol., 2013, 31, 108-115).

In the present invention, the cells constituting the spheroid may be used without limitation. For example, cells constituting the spheroid include disease-related cells, preferably tumor cells, or the like, but are not limited thereto.

In the present invention, the spheroid array may be prepared by various methods known in the art. Nonlimiting examples of the methods include a method of forming an array by producing one spheroid for each compartment such as a hanging drop or low-attachment well plate, and a method of forming a spheroid array by producing large-scale spheroids in one medium such as a spinner flask, a microwell array, and a hanging drop microarray.

In one embodiment of the present invention, a spheroid array was prepared by culturing spheroids in a microwell array and then flipping the array, or using a hanging drop microarray having a plurality of holes at the bottom, but is not limited thereto.

The hanging drop microarray prepared and used in one embodiment of the present invention was the hanging drop microarray having a plurality of holes at the bottom disclosed in International Patent Publication No. WO 2015/129263. The hanging drop microarray having a plurality of holes at the bottom has a configuration in which cells aggregate in the droplets formed through the plurality of holes to form a spheroid in the form of a hanging drop. The hanging drop microarray having such a structure advantageously directly contacts the bottom of the contact-based droplet trapping structure array of the present invention, thereby trapping spheroid droplets and transferring spheroids.

Therefore, in the present invention, the spheroid array is preferably a flipped microwell array or a hanging drop microarray, more preferably a hanging drop microarray, but is not limited thereto.

In one embodiment of the present invention, the spheroid array is manually brought into contact with the contact-based droplet trapping structure array. A spacer formed by cutting PMMA was used to easily adjust the distance between the two arrays.

Therefore, in the present invention, the droplet transfer device or the spheroid transfer device may further include a spacer.

In the present invention, the spheroid array contacts the concave plateau of the contact-based droplet trapping structure array.

In one embodiment of the present invention, the two arrays are brought into contact with each other manually, but those skilled in the art will appreciate that the contact between the drop array (or spheroid array) and the contact-based droplet trapping structure array may be automatically performed.

When the droplet transfer device or the spheroid transfer device of the present invention is automated, an X-Y axis alignment and movement device for aligning each array, a Z-axis movement device for contact and release, a control unit for controlling each movement device, and a storage device for various settings may be included without limitation.

As in one embodiment of the present invention, upon contact of the two arrays, spheroids and droplets are transferred and trapped by gravity from the upper array to the lower array. As in another embodiment of the present invention, when the contact-based droplet trapping structure array that traps the spheroid-droplet is brought into contact with the drop array containing a reagent such as PBS at the bottom thereof, reagent treatment can be performed without spheroid transfer.

Therefore, the droplet transfer device or the spheroid transfer device of the present invention may further include one or more drop arrays and may further include a rotary motion device to change the position of each array.

In another aspect, the present invention is directed to a droplet transfer method, including:

(a) bringing a drop array into contact with the contact-based droplet trapping structure array of the present invention; and

(b) releasing the contact between the drop array and the contact-based droplet trapping structure array to trap droplets in a concave plateau of the contact-based droplet trapping structure array.

In the present invention, in step (a), the drop array is brought into contact with the concave plateau of the contact-based droplet trapping structure array on the top of the contact-based droplet trapping structure array.

In the present invention, the method may further include aligning the drop array and the contact-based droplet trapping structure array of the present invention before step (a).

In the present invention, the droplet may contain a spheroid, and in this case, the spheroids are transferred to the concave plateau of the contact-based droplet trapping structure array by gravity along with the trap of the droplets. In the present invention, the drop array may be a spheroid array including droplets containing spheroids.

In another aspect, the present invention is directed to a droplet contact-based spheroid transfer method, including:

(a) bringing a spheroid array into contact with the contact-based droplet trapping structure array of the present invention; and

(b) releasing the contact between the spheroid array and the contact-based droplet trapping structure array to transfer droplets containing spheroids to the concave plateau of the contact-based droplet trapping structure array.

In the present invention, the method may further include aligning the drop array and the contact-based droplet trapping structure array of the present invention before step (a).

In the present invention, in step (a), the drop array is brought into contact with the concave plateau of the contact-based droplet trapping structure array on the top of the contact-based droplet trapping structure array.

In the present invention, the droplets containing spheroids are each separated from the spheroid array by the spheroid transfer method to form an isolated spheroid droplet array, and the spheroid droplet of the isolated spheroid droplet array is physically isolated, which allows for treatment with different conditions or reagents.

Therefore, in another aspect, the present invention is directed to a method for manufacturing a spheroid array using the spheroid transfer method. In the spheroid array of the present invention, each spheroid is separated into an isolated environment.

Therefore, in another aspect, the present invention is directed to an isolated spheroid array produced by the method of manufacturing the spheroid array.

In one embodiment of the present invention, it was found that the contact-based droplet trapping structure array and the spheroid transfer method of the present invention can be utilized in various applications such as medium exchange, treatment with various reagents, chemical staining, immunostaining, and clearing.

Therefore, the contact-based droplet trapping structure array of the present invention can be useful not only for the transfer of spheroids, but also for simultaneous reagent treatment of the isolated spheroid droplet arrays.

In another aspect, the present invention is directed to a method of treating spheroids with a reagent, including:

(a) bringing a cultured spheroid array into contact with the contact-based droplet trapping structure array;

(b) releasing the contact between the cultured spheroid array and the contact-based droplet trapping structure array to produce an isolated spheroid droplet array; and

(c) bringing the isolated spheroid droplet array into contact with a reagent-loaded array.

In the present invention, steps (a) and (c) may further include arranging each array before contact.

In one embodiment of the present invention, it was found that when washing is performed without transferring the spheroids while repeatedly bringing the drop array loaded with washing buffer (PBS) into contact with the isolated spheroid droplet array on the top of the isolated spheroid droplet array and then releasing the contact, the amount of a first reagent transferred to a second reagent greatly decreased depending on the number of repetitions.

Therefore, in the present invention, the method may further include, before step (c), (b′) washing, while repeatedly bringing the drop array loaded with washing buffer (PBS) into contact with the isolated spheroid droplet array at the bottom of the drop array and then releasing the contact.

In the present invention, the washing may be performed at least one time.

In the present invention, the washing may be performed at least one time. In the present invention, the amount of the first reagent transferred to the second reagent decreases as the number of washes increases. In an embodiment of the present invention, when washing is performed 20 times or more, the first reagent is transferred in an amount of less than 0.1%.

In the present invention, the spheroids may be transferred from the upper array to the lower array by gravity.

Therefore, in the present invention, in step (c), the spheroids are treated with reagents by bringing the isolated spheroid droplet array into contact with the top or bottom of the reagent-loaded array depending on whether or not the spheroids need to be transferred.

In the present invention, the reagents may be used without limitation. For example, in one embodiment of the present invention, various types of reagents such as culture media, washing buffers such as PBS, formaldehyde solutions, chemical staining reagents, antibodies, and antibiotics are used, but are not limited thereto.

The spheroid transfer method of the present invention can be easily used for medium exchange.

In another aspect, the present invention is directed to a method of exchanging a spheroid medium, including:

(a) bringing a cultured spheroid array into contact with the contact-based droplet trapping structure array;

(b) releasing the contact between the cultured spheroid array and the contact-based droplet trapping structure array to produce an isolated spheroid droplet array; and

(c) bringing the isolated spheroid droplet array into contact with a medium-loaded drop array to transfer spheroids.

In the present invention, steps (a) and (c) may further include arranging each array before contact.

In the present invention, the method may further include washing before step (c) to minimize contamination between media.

Therefore, the method may further include, before step (c), (b′) washing, while repeatedly bringing the isolated spheroid droplet array into contact with the bottom of the drop array loaded with washing buffer and then releasing the contact.

In the present invention, in step (c), the isolated spheroid droplet array is brought into contact with the medium-loaded drop array on the top of the medium-loaded drop array. At this time, the spheroids are transferred by gravity to the medium-loaded drop array.

Hereinafter, the present invention will be described in more detail with reference to examples. However, it will be obvious to those skilled in the art that these examples are provided only for illustration of the present invention and should not be construed as limiting the scope of the present invention.

### EXAMPLE

### Example 1: Materials and Methods

### Example 1-1: Cells and Reagents

PlasCLEAR (Asiga, Australia));

Polyurethane release agent (Nabakem, Korea);

Poly(dimethylsiloxane) (PDMS) monomer and curing agent (Dow Corning, USA);

Poly(methyl methacrylate) (PMMA) plate (YM Tech, Korea);

16% w/v formaldehyde solution and Image-iT™ red hypoxia reagent (Thermo Fisher Scientific, USA);

Triton X-100 (Junsei, Japan). BSA (bovine serum albumin) (Santa Cruz Biotechnology Inc., USA). PBS (phosphate buffered saline), DMEM (Dulbecco's modified Eagle's medium), FBS (fetal bovine serum), and penicillin/streptomycin (P/S) (Corning, USA);

Human breast cancer cell line BT-474 (KCLB No. 60062) (Korean Cell Line Bank (KCLB; Seoul, Korea);

Calcein-AM, EthD-1 (ethidium homodimer-1), and Hoechst 33342 (Invitrogen, USA);

Mouse anti-human hypoxia-inducible factor-1 alpha antibody (HIF-1α antibody) (No. 610959) (BD Biosciences, USA);

Rabbit anti-E-cadherin antibody (ab40772), Alexa Fluor 594-conjugated goat anti-rabbit IgG H&L antibody (ab150080), and Alexa Fluor 488-conjugated goat anti-mouse IgG H&L antibody (ab150113) (Abcam, United Kingdom); and other compounds and reagents (Sigma-Aldrich, USA).

### Example 1-2: Production of Droplet Contact-Based Spheroid Transfer (DCST) Device

Replica molds of the DCST device, drop array chip, and pillar array chip were designed using Autodesk Inventor 2018 and 3D printing was performed using a 3D printer (Pico2 HD; Asiga, Alexandria, Australia) based on PlasCLEAR resin. The printed mold was immersed in ethanol and sonicated for 20 minutes. After drying the mold, both surfaces thereof were irradiated with UV light for 3 minutes to cure the uncured resin. To facilitate the PDMS release process, the mold was treated with 02 plasma for 1 minute and then exposed to 1H,1H,2H,2H-perfluorooctyl-trichlorosilane under vacuum. The treated mold was further coated with a polyurethane release agent before use.

The DCST device was produced using PDMS molding in the mold. An uncured PDMS mixture (monomer: curing agent=10:1) was poured into a mold and cured in a convection oven at 85° C. for 2 hours. The device separated from the mold was immersed in distilled water, autoclaved and then dried in a convection oven. Spacers and support pillars were produced by cutting PMMA using a laser cutter (C40-60 W; Korea).

### Example 1-3: Cell and Spheroid Culture

BT-474 cells were allowed to stand in DMEM supplemented with 10% FBS and 1% P/S in a humidified incubator at 37° C. and 5% CO₂. Culture medium was exchanged every 2 days and cell confluency was maintained below 80%. To form spheroids in a hanging drop manner, a cell suspension was prepared at the desired concentration and seeded in a droplet volume of 25 μL into the wells of the DAC. After loading the droplet, the DAC was placed on two PMMA columns attached to the culture plate. In order to prevent droplet evaporation, the plate was filled with PBS and another plate filled with PBS was placed on top of the plate containing DAC. To form spheroids with an ultra-low attachment plate, the cell suspension prepared at the desired concentration was seeded in each well of a 384-well spheroid microplate (Corning) and the liquid volume was adjusted to 50 μL. The culture medium was exchanged every 2 days.

### Example 1-4: Measurement of Amount of Remaining Reagent in Droplets

Residual reagents in the droplets on the pillar array chip (PAC) were calculated in the same manner as in previous studies (H. Kim et al., Biomicrofluidics, 2018, 12, 044109). A volume calibration curve was obtained by measuring the peak absorbance at 406 nm of an erioglaucine solution formed by mixing 0.5 to 5 μL of a 1 mM erioglaucine solution with 100 μL of PBS. The droplets were transferred to a 96-well plate filled with 100 μL of PBS and then the volume and concentration of the droplets were measured and calculated using a microplate spectrophotometer (SpectraMax 250; Molecular Devices, Sunnyvale, Calif., USA). The residual amount of the first reagent that is transferred by bringing the PAC into contact with the second reagent droplet of the DAC was measured using a spectrophotometer (Nanodrop 2000; Thermo Fisher Scientific). After obtaining the volume calibration curve, the amount and concentration of the first reagent solution transferred to the second reagent droplet were calculated from 2 μL of the droplet.

### Example 1-5: Live/Dead and Hypoxia Imaging of Spheroids

Cell viability was evaluated using a conventionally known live/dead fluorescence staining method. Spheroids were stained using 4 μM calcein-AM and a 4 μM EthD-1 solution at 37° C. for 1 hour. After washing with medium, fluorescence images were obtained using an inverted microscope (IX51; Olympus, Tokyo, Japan).

For hypoxic imaging, the spheroids were treated with 10 μM Image-iT™ red hypoxia reagent at 37° C. for 24 hours, the solution was replaced with a culture medium, spheroids were cultured for 48 hours and images were obtained with a microscope.

### Example 1-6: Immunostaining of Spheroids

To analyze protein markers of spheroids, fixation was performed in 4% formaldehyde solution for 30 minutes, and permeabilization was performed in 0.1% Triton X-100 solution for 1 hour, followed by blocking in 5% BSA solution at room temperature for 30 minutes. Spheroids were first treated in an orbital shaker with mouse anti-HIF-la and rabbit anti-E cadherin monoclonal antibodies at 4° C. overnight. Then, the spheroids were treated with goat anti-mouse and anti-rabbit polyclonal antibodies at 37° C. for 2 hours. Finally, nuclei were counterstained using a 5 μg/ml Hoechst 33342 solution at room temperature for 10 minutes. In the case of the DCST method, the DAC and PAC used in the immunostaining step were pretreated with a 5% BSA solution for 30 minutes to prevent adhesion of spheroids.

### Example 1-7: Optical Clearing of Spheroids

Previously reported RTF, ScaleSQ(0), modified ScaleS, and FOCM methods were performed for clearing of immunostained spheroids.

E. Boutin et al., Sci. Rep., 2018, 8, 11135. 43; X. Zhu et al., Proc. Natl. Acad. Sci. U.S.A., 2019, 166, 11480-11489)

RTF

The RTF solution was prepared by mixing triethanolamine, formamide and distilled water as previously reported (T. Yu et al., Sci. Rep., 2018, 8, 1964. 41), and the spheroid solution was sequentially treated with RTF-R1, RTF-R2 and RTF-R3 solutions at room temperature for 30 minutes, 1 hour and 1.5 hours, respectively. The spheroids were immersed in RTF-R3 solution and placed in confocal dishes prior to imaging.

ScaleSQ(0)

ScaleSQ(0) and ScaleS4(0) solutions were prepared as previously reported (H. Hama et al., Nat. Neurosci., 2015, 18). To prevent urea precipitation, the ScaleSQ(0) solution was stored above 30° C. until use. Spheroids treated using the ScaleS-based method were post-fixed after immunostaining in accordance with previously reported recommendations. The spheroids post-fixed with 4% formaldehyde solution for 30 minutes were sequentially treated with ScaleSQ(0) and ScaleS4(0) at 37° C. for 2 hours. After optical clearing, the spheroids were immersed in ScaleS4(0) solution and placed in a confocal dish.

Modified Scales.

A ScaleS4 solution was prepared in accordance with a previously known method by mixing D-(−)-sorbitol, glycerol, urea, Triton X-100, and dimethyl sulfoxide (DMSO) in distilled water. At this time, the Triton-X 100 was set at 0.1%. Modified ScaleS clearing was performed in accordance with the protocol suggested by Boutin et al. Sci. Rep., 2018, 8, 11135. The post-fixed spheroids were treated with ScaleS4 solution at 37° C. for 12 hours and then placed in confocal dishes.

FOCM

The FOCM solution was prepared by mixing 30% (w/v) urea, 20% (w/v) D-(−)-sorbitol, and 5% (w/v) glycerol with DMSO, which is the same solution as for brain slice clearing conditions (X. Zhu et al., Proc. Natl. Acad. Sci. U.S.A, 2019, 166, 11480-11489). Spheroids were treated with FOCM solution at room temperature for 10 minutes and then placed in confocal dishes.

All cleared samples were transferred to a confocal dish using the DCST device of the present invention and observed with a confocal laser scanning microscope (LSM 880; Carl Zeiss, Oberkochen, Germany) at the KAIST Analysis Center (Daej eon, Korea).

### Example 1-8: Statistical Analysis

Data are expressed as mean±standard deviation.

Data from the three groups were compared using one-way ANOVA with Games-Howell post hoc tests in IBM SPSS statistics. p values >0.05, <0.05 and <0.01 were considered “not significant (n.s.)”, “statistically significant (*)” and “very significant (**)”, respectively.

### Example 2: Design and Operation of Droplet Contact-Based Spheroid Transfer (DCST) Device

Sequential reagent treatment was performed by repeatedly transferring the spheroid array using a DCST device including a drop array chip (DAC) and a pillar array chip (PAC) (FIG. 1 in part A thereof). In an embodiment of the present invention, the DCST device includes a DAC, a PAC, and a spacer to adjust the contact spacing thereof and an exemplary design thereof is shown in part B of FIG. 1.

The sizes of DACs and PACs were adjusted depending on a general 384-well plate, and the sizes of the arrays used in Examples were 2×2 (for clearing), 4×4 (for characterization), and 5×10 (for spheroid culture and immunostaining), designed in various ways as needed.

The DAC used to form spheroid culture or reagent droplets was designed to be 3 mm in diameter and height, and to contain droplets of up to 25 μL. The bottom of the DAC was designed to be round to prevent air bubbles from forming at the edges. A cell suspension was loaded and cultured on the DAC to culture spheroids or reagents were loaded in an empty DAC to change the droplet environment of the spheroid array. The method of loading reagents in the DAC and then transferring spheroids using DCST does not include pipetting and thus there is little risk of loss or damage of spheroids during the reagent replacement process.

A pillar array chip (PAC) was designed to be used to stably retain and transfer spheroids. The pillar array chip was produced as three types, in other words, a flat plateau having a truncated cone shape, a concave plateau having a concave top, and a pillar at the top thereof, including microwalls having air channels (i.e., a concave plateau with air channels).

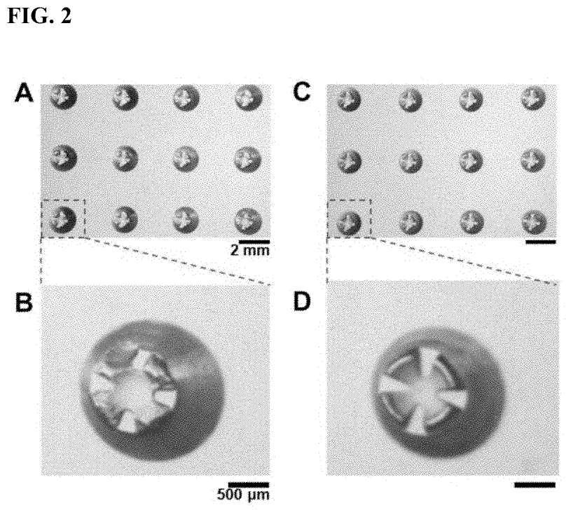

As previously reported by the present inventors, the DAC and PAC molds were produced by laser cutting of PMMA, but in the embodiment of the present invention, the DAC and PAC molds were manufactured using 3D printing based on a photocurable resin (FIG. 1 in part C thereof). Since the PAC manufactured in the embodiment of the present invention has a concave top structure and a complicated structure of air channels, a PDMS-based device with a complicated structure was produced by treating with silane in the manufacturing process (FIG. 2). Because 3D printing has a high degree of design freedom, 3D printed DCST devices enable a design suitable to culture, treatment and management of spheroids, as compared to those produced using previously reported methods (H. Kim et al., Biomicrofluidics, 2018, 12, 044109) or conventional photolithography.

FIG. 1 in part D thereof illustrates details of the spheroid transfer and reagent treatment methods using the DCST device according to the present invention. Specifically, the spheroids cultured in the DAC come in contact with the PAC and move to the top of the PAC, and the spheroids on the top of the PAC come into contact with a washing buffer or another DAC loaded with reagents. In this state, reagent treatment is possible. In the above process, the spheroid moves or remains on the DAC or PAC positioned thereunder by gravity, and thus can be designed and used to move or remain on the desired array chip.

### Example 3: Confirmation of Spheroid Transfer Efficiency of Droplet Contact-Based Spheroid Transfer (DCST) Device

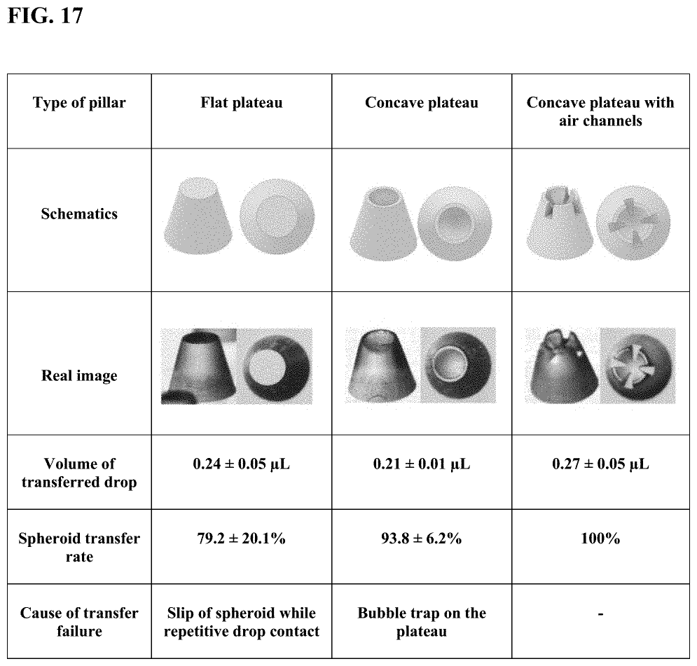

In order to design the pillar design of the PAC that can most stably hold and transfer the spheroids, spheroid transfer was performed using three designs of PAC with a BT-474 spheroid with a diameter of 450 μm and a top width of 1 mm (FIG. 17).

In a previous report (H. Kim et al., Biomicrofluidics, 2018, 12, 044109), the pillar with a flat top developed by the present inventors had a spheroid transfer rate of about 80%. When the DAC and the PAC, including a pillar having a flat top come into contact, a droplet with a low height moves to the top of the pillar. When the height of the spheroid is greater than that of the droplet, the spheroid is not stably fixed, especially when the column is slightly tilted or external force is applied, for example, repeated contact occurs, due to the flat top, the droplets and the spheroids flow down along the side surface of the pillar, which was lost.

The pillar with a concave top was designed such that the height of the droplet transferred by the contact between the DAC and PAC was formed higher than that of the pillar with a flat top and the spheroid was located in the center. The PAC produced as a pillar with a concave top increased spheroid transfer rate by about 10%, but the spheroid was not properly transferred when air was trapped between the concave structure and the droplet.

To overcome this problem, the PAC using a concave structure and a pillar with air channels to allow air trapped between the droplets to escape was capable of accommodating the largest volume of droplets most stably and exhibited a spheroid transfer rate of 100%.

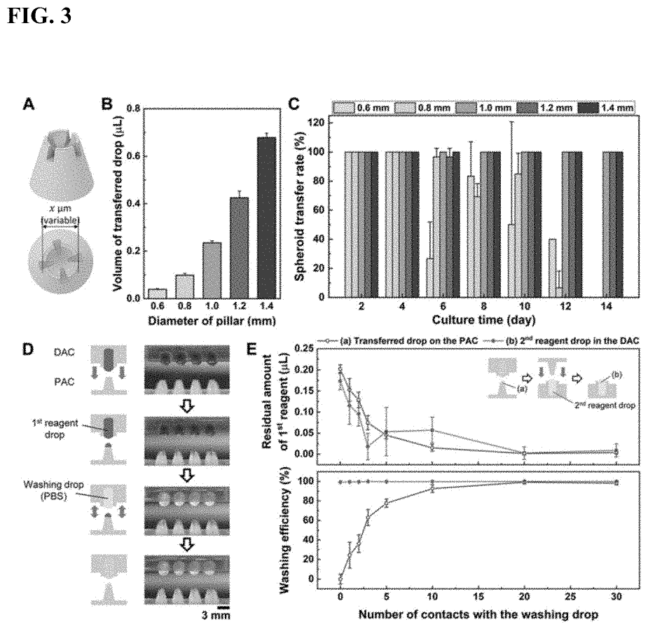

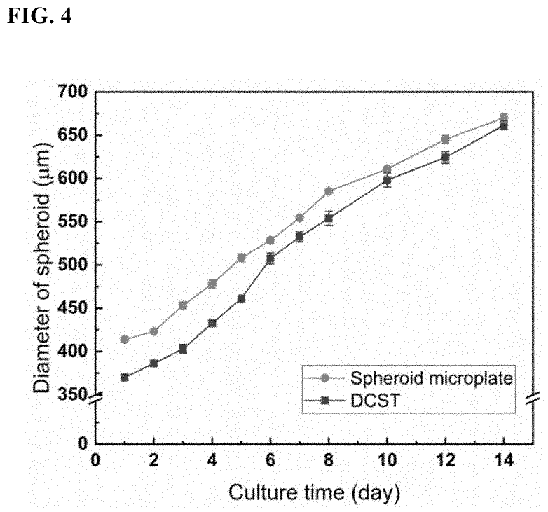

Next, the correlation between the spheroid transfer rate and the diameter of the column and the size of the spheroid was determined (FIG. 3 in parts A to C thereof). BT-474 cells were seeded at a concentration of 4,000 cells/droplet and cultured using a DCST device for 2 weeks (FIG. 4).

Compared to spheroids cultured in conventional spheroid microplates under the same conditions, the spheroids cultured using the DCST device were initially small in size, but had a high growth rate and grew to a size that was the same as spheroids cultured in spheroid microplates on day 14. The volume of the droplets transferred to the pillar increased in proportion to the diameter of the pillar (FIG. 3 in part B thereof) and the volume of the droplet transferred to the pillar with a diameter of 1.4 mm was 0.7 μL. Small spheroids were well transferred at all pillar diameters, but the transfer rate to small-diameter pillars gradually decreased as the size of the spheroids increased (FIG. 3 in part C thereof). On the other hand, pillars with a diameter of 1 mm or more had a 100% transfer rate even for spheroids with a size of about 660 μm cultured for 14 days.

The above result means that, when designing the size of the pillar for spheroid transfer, the pillar should be designed to have a predetermined margin (at least 300 μm or more) from the diameter of the spheroid. When the diameter of the spheroid is equal to or higher than the diameter of the pillar, the spheroid is not transferred even when the DAC contacts the PAC. Therefore, in the following example, an experiment was conducted using a PAC having a concave pillar having an air channel having a top diameter of 1 mm.

### Example 4: DCST-Based Reagent Treatment Method

A DCST device-based reagent treatment method to treat spheroids independently transferred by PAC with reagents was performed. A washing step was introduced between respective steps to prevent cross-contamination between reagents. An erioglaucine solution as the first reagent and PBS as the washing buffer were used to determine the washing efficiency using the DCST device (FIG. 3 in part D thereof). First, the DAC loaded with the first reagent was brought into contact with the PAC and a droplet of the first reagent was transferred to the top of the PAC. Then, the DAC loaded with PBS was repeatedly brought into contact with the PAC to which the first reagent droplet was transferred, and finally the volume and concentration of the first reagent in the washed PAC droplet were measured. After the washing step, the DAC loaded with the second reagent was brought into contact with the PAC, and the concentration of the first reagent was measured in the DAC loaded with the second reagent.

As shown in FIG. 3 in part E thereof, as the number of contacts in the washing step increased, the volume of the first reagent gradually decreased and the washing efficiency increased. The washing efficiency converged when the number of contacts with the DAC loaded with the washing buffer was 20 or more. The amount of the first reagent transferred to the DAC loaded with the second reagent was lower than the amount of the first reagent present on the PAC even when the washing step was not performed, and it decreased as the number of contacts increased. The amount converged when the number of contacts exceeded 20 times. On the other hand, the washing efficiency in the secondary reagent droplet started at 99.3% when the number of contacts between the droplet on the PAC and the washed droplet was 0 and converged on 99.99% when the number of contacts was 20 or more. This is because the droplet volume transferred to the PAC is less than 1% of the droplet volume of the DAC. Therefore, when the spheroids that have undergone sufficient washing are transferred to the secondary reagent droplets, the concentrations of the primary and secondary reagents become 0.01% and 99.3% of the initial concentrations, respectively.

### Example 5: Experiment to Compare Variance Between Users

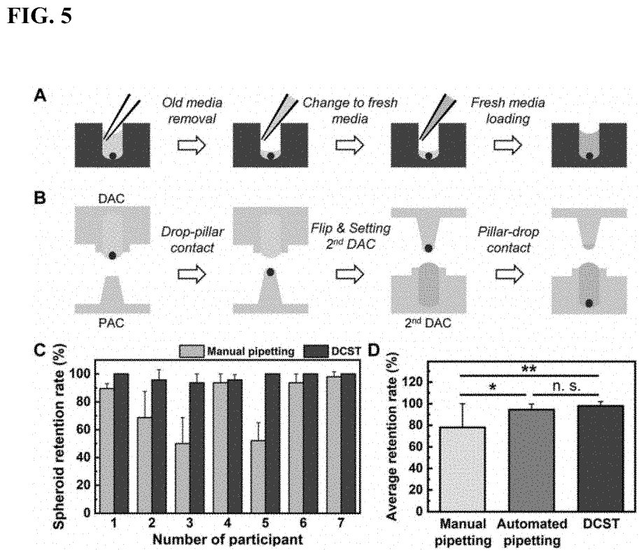

Since the transfer of spheroids using the conventional manual pipetting method has a very great variation between users depending on the skill level of the user, the variation between users in case of using the DCST device was compared. The spheroid culture medium was exchanged with fresh medium using the reagent exchange step of Example 4. Seven experimenters who had never handled DCST devices or spheroid microplates were recruited. FIG. 5 in part A thereof shows a medium exchange method using pipetting and FIG. 5 in part B thereof shows a medium exchange method using a DCST device. Each experiment was performed using 16 spheroids and was repeated three times independently. In addition, in order to compare the method using the DCST device with the method using the automated pipetting platform, spheroids having the same conditions were prepared in a 96-well spheroid microplate and the medium was exchanged using an automated pipetting platform (12-channel VIAFLO electronic pipette integrated with an ASSIST pipette adapter; Integra Biosciences AG, Zizers, Switzerland). The retention rate of spheroids and the volume of residual droplets depending on the height of the pipette tip from the bottom are shown in FIG. 6. Up to 24 spheroids were used in the automatic pipetting experiment.

As shown in FIG. 5 in parts C and D thereof, the retention rate of spheroids when using the medium change using the DCST device in the data of all experimental participants was significantly higher than when the medium was changed using manual pipetting (FIG. 5 in part C thereof). In addition, the method using the DCST device showed significantly smaller deviations than the manual pipetting method. The method using the DCST device exhibited an average retention rate of spheroids of 97.92% and a standard deviation of 4.11% compared to the overall average, and the method using manual pipetting exhibited an average retention rate of 77.98% and a standard deviation of 21.97% (FIG. 5 in part D thereof). In addition, the method using the DCST device exhibited a coefficient of variation of 4.20%, whereas the manual pipetting method exhibited a coefficient of variation of 28.18%, which means that the method using the DCST device exhibits a higher spheroid retention rate than manual pipetting, regardless of the user, and when two or more medium exchanges or reagent treatments are further performed, the difference in average retention rate and standard deviation between the two methods becomes larger.

In addition, even an automatic pipetting platform exhibited an average retention rate of 94.44%, a standard deviation of 5.20%, and a coefficient of variation of 5.50%, which are slightly lower efficiency than DCST. In the automatic pipetting platform, the optimal height of the pipette tip to remove the residual liquid as much as possible while minimizing the loss of spheroids was 1.3 mm. In this case, the remaining liquid was 13.63 μL, corresponding to a residual rate of about 7%. However, the residual liquid volume was about 1% in the DCST device using a 1 mm pillar as described above. This means that two or more washing steps are required in the automated pipetting platform to achieve the same effect as adding one washing step between reagent changes in the method using the DCST device. The DCST showed somewhat better spheroid retention and deviation than the automated pipetting platform, but a much smaller residual liquid volume than the automated pipetting platform.

### Example 6: Chemical Staining and Immunofluorescence Staining Using DCST

Live/dead staining, hypoxic staining, and immunostaining of spheroids were performed using the DCST device to determine whether or not a platform based on the DCST device could act as a platform for biological analysis of spheroid arrays. BT-474 cells were seeded in the DAC at an initial inoculation concentration of 4,000 cells per droplet and cultured for 14 days.

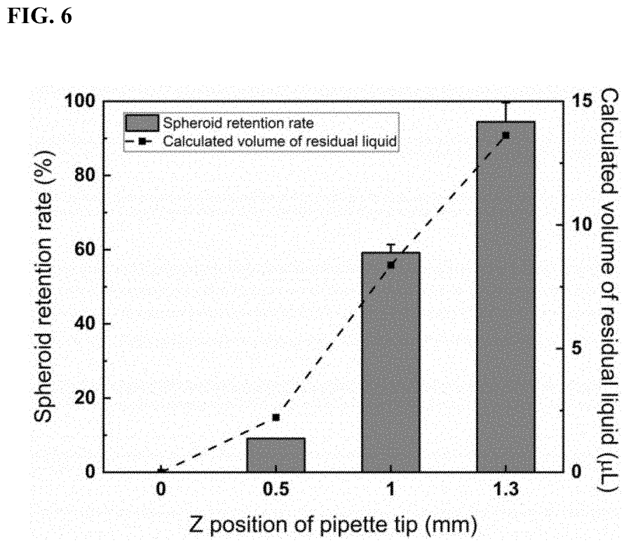

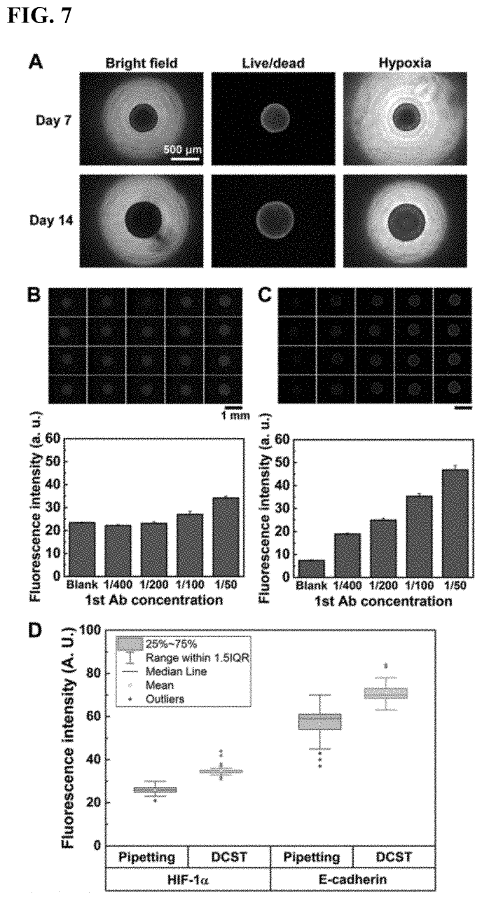

Live/Dead staining and hypoxic staining of spheroid arrays using the compound can all be performed using the DCST device (FIG. 7 in part A thereof). After 2 weeks, spheroids exhibited necrotic and hypoxic cores, as previously reported. The hypoxic area appeared larger and more distinct than the necrotic core. On day 7 of culture, hypoxia was not expressed and necrotic centers were not present in spheroids having a size of 500 μm. In consideration of previous studies that spheroids cultured in a device having a bottom capable of exhibiting high oxygen permeability had fewer necrotic cores and hypoxic parts, the hanging drop system has higher oxygen permeability than conventional well plates or other microfluids.

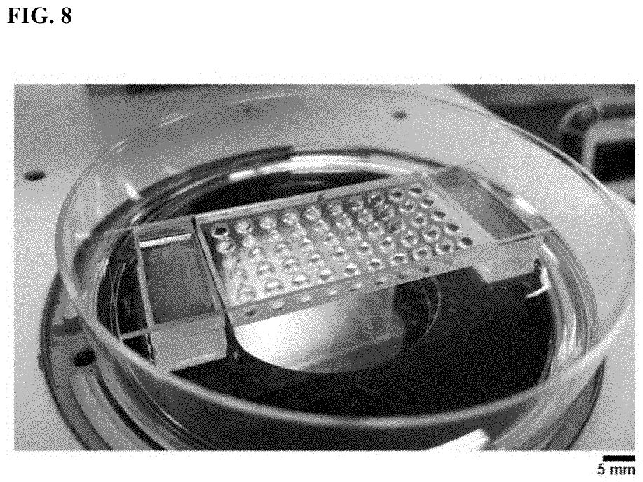

Next, in order to determine whether or not the target protein can be well labeled when immunostaining is performed using the DCST device, HIF-1α as a hypoxia marker and E-cadherin as a cell-cell adhesion marker were immunostained as targets. Spheroids were immunostained after fixation, permeabilization and blocking using DCST. In the entire immunostaining process including the pretreatment, BSA-coated DAC and PAC were used because spheroids were attached to the device after the fixation step when the coating was not applied (FIG. 8). When immunostaining was performed using an uncoated device, spheroids were lost because the spheroids were not subjected to the final process. Therefore, whether or not the device was coated with the BSA solution while preventing the formation of air bubbles was determined. To prove that multiple conditions can be simultaneously applied to each spheroid in one device, the spheroid array was immunostained by configuring the DAC with five different conditions for each row of the spheroid array. The intensity of fluorescence of all antibody types tested increased with an increase in the concentration of the primary antibody (FIG. 7 in parts B and C thereof). In addition, the fluorescence intensity tended to appear brighter in E-cadherin than in HIF-1α. When the primary antibody concentration was 1/100, the HIF-1α started to differ from the blank value, and when the primary antibody concentration was 1/50, the difference was clearly distinguished. On the other hand, E-cadherin was different from the blank value when the primary antibody concentration was 1/400.

To determine staining uniformity in the spheroid array, spheroids in the DCST device and conventional spheroid microplates were immunostained using DCST and manual pipetting methods, respectively (FIG. 7 in part D thereof). Spheroid arrays immunostained using the DCST device were stained more uniformly compared to the manual pipetting method in all tested conditions, and the fluorescence intensity thereof was also higher than when immunostaining was performed on spheroid microplates using the manual pipetting method. The coefficients of variation of BT-474 spheroids stained by manual pipetting were 5.52% for HIF-1α and 9.59% for E-cadherin. The spheroids stained using DCST had lower coefficients of variation under each condition of 2.49% and 4.85%, respectively. The reliability of the data increases when the coefficient of variation is less than 5%, which indicates that the DCST platform is applicable as a reliable immunostaining platform.

### Example 7: Clearing (Transparency) of Spheroids Using DCST Device

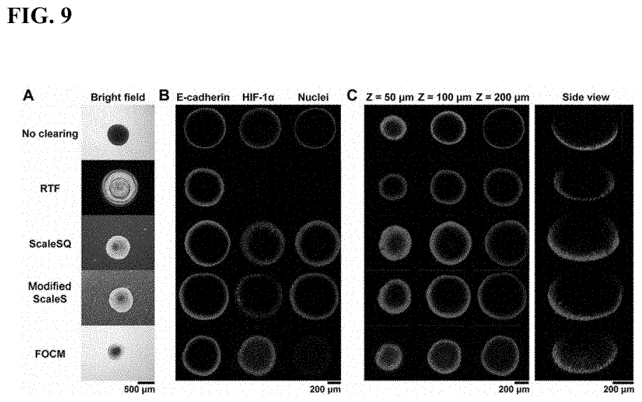

Whether or not clearing using the DCST-based device of the present invention is possible as the last step of whole spheroid imaging was determined. After the spheroid array was washed with PBS, it was placed on a PAC and brought into contact with a clearing solution-loaded DAC. Due to the density difference between water (or PBS) and the clearing solution, the spheroids floated to the top of the droplet immediately after contact and then settled again as the liquid inside the spheroids was replaced with the clearing solution. When spheroids were cultured in the form of hanging drops in the clearing solution, they were cultured without placing PBS on the bottom of the culture vessel to prevent an increase in drop volume due to the difference in evaporation rate. The result of clearing the spheroids is shown in FIG. 9. In all clearing methods, the spheroid became more clarified, and fluorescence signals of E-cadherin, HIF-1α, and nuclei were observed to be derived from inside the spheroid. In simple comparison, the light penetration depth of non-clarified spheroids was about 40 μm, whereas the penetration depth of the clarified spheroids was about twice that or more. However, some fluorescence signals were observed to be significantly attenuated in spheroids cleared by the RTF and FOCM methods. RTF is an improved method of ClearT2 and it was reported that the fluorescence signal of the nucleus is weakened in ClearT2 samples. Therefore, it is considered that the signal is weakened for the same reason. FOCM is an improved method of ScaleS and quick clearing is possible by changing the solvent from water to DMSO, which means that there may be present a harsh environment where delipidation and hyperhydration processes occur quickly. In addition, in ScaleSQ(0) and the modified ScaleS, post-fixation was performed immediately after immunostaining according to the manual, but in the FOCM, it was considered that the fluorescence signal was weakened because fixing was not performed due to invisibility. Nonetheless, it was found that clearing the spheroids helps to facilitate imaging inside the spheroids and can be an essential technique for position-specific expression analysis of target markers in whole spheroids.

### Example 8: Design of Different Types of DCST Devices

### Example 8-1: Hanging Drop Microarray (HDMA)

In the DCST prepared in Example above, spheroids were cultured using a drop array chip (DAC) in the form of a bottom-closed well plate and the DAC of the present invention was flipped and brought into contact with the column array chip (PAC) to transfer the spheroids to the top of the column in the PAC. However, the DCST device using the DAC having this structure cannot eliminate the possibility of damage or loss of spheroids, for example, a phenomenon in which the solution containing spheroids does not form a drop and flows down in the process of flipping the DAC, depending on the viscosity of the spheroid culture solution, the size and material of the well, and the proficiency of the user.

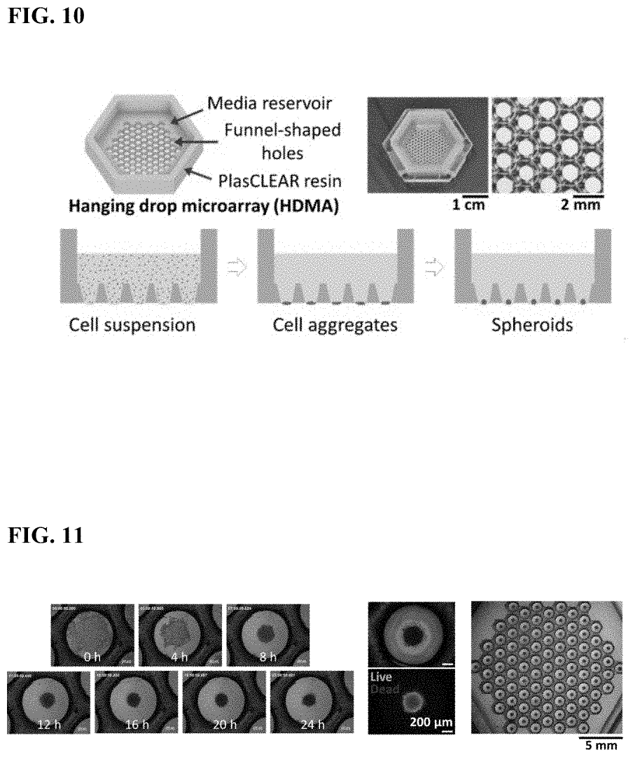

Therefore, the present inventors manufactured a hanging drop microarray having a plurality of holes at the bottom in order to bring the droplets of the drop array into contact with the PAC without a separate flip process (Hanging Drop Microarray; HDMA), as disclosed in International Patent Publication No. WO 2015/129263 (FIG. 10).

As shown in International Patent Publication No. WO 2015/129263 and FIG. 10, the HDMA has a structure including a plurality of fine holes at the bottom thereof and the HDMA is spaced from the bottom by a predetermined distance to prevent the holes at the bottom thereof from being clogged by solid or liquid surfaces. When the cell suspension is injected into the vessel, the cells are settled due to gravity over time to form spheroids in the bottom holes.

FIG. 11 shows the result of the generation of spheroids in the HDMA having a plurality of holes at the bottom thereof. The culture of spheroids was performed by replacing the culture medium at intervals of one day. Over time, the cells sinking into the pores interact with each other to form dense spheroids. In this process, the survival rate of cells is maintained high. In addition, as many spheroids are generated as the number of holes, and one spheroid exists in each hole. Furthermore, as described above, the hanging drop system has reduced necrotic cores and hypoxic areas due to the relatively higher oxygen permeability than the spheroids prepared using the well plate of Example 6 because spheroids are formed at the interface between droplets and air.

### Example 8-2: Design and Manufacture of Microarrays for Trapping Droplets Having Various Types of Droplet Trapping Structures

Using the concave pillar microarray (CPMA) based on a concave pillar having a concave top including an air channel, which exhibits an excellent effect in Example 3, the array chip was manufactured by designing the distance and size so as to contact the micropores of the HDMA prepared in Example 8-1 (FIG. 12).

Furthermore, since the transfer of spheroid droplets using a concave pillar-based microarray occurs by contact with a concave structure including walls substantially including air channels formed at the top of the pillar, the pillar is removed and microwalls including air channels are formed on the plane to produce a new type of droplet-trapping array chip (FIG. 13).

As another example of an array chip for trapping droplets, the array chip for trapping droplets can be manufactured by designing a portion where a droplet is placed to be hydrophilic and the other portion to be hydrophobic so as to stably maintain the size of droplets without a microstructure.

### Example 8-3: Spheroid Transfer Method Using DCST Device

A droplet contact-based spheroid transfer (DCST) device was produced using the hanging drop microarray and various droplet trapping microarrays.

FIG. 14 shows a method of separating all (FIG. 14 in part A thereof) or part (FIG. 14 in part B thereof) of spheroids prepared in the hanging drop microarray through the droplet contact-based spheroid transfer method using the concave pillar microarray (CPMA) of the present invention into isolated droplet arrays. Specifically, the position of the hole of the container and the top surface of the pillar of the device are arranged and then the gap between the two is narrowed so that the liquid in the container comes into contact with the top surface of the pillar. Then, when the gap between the two is widened again to return to a non-contact state, droplets made of the same liquid as the liquid present in the container are formed on the top surface of the pillar, and the spheroid remains in the droplet located on the top surface of the pillar due to gravity. Based thereon, a spheroid array formed in large quantities and linked under the same liquid environment can exist in the form of droplet arrays separated from each other. As a result, spheroid droplet arrays can be formed quickly and efficiently, and the spheroid arrays can be simultaneously treated under different spheroid types, concentrations, and combinations later.

### Example 8-4: Confirmation of Spheroid Transfer Using Droplet Contact-Based Spheroid Transfer (DCST) Device

The transfer of spheroids using the droplet contact-based spheroid transfer device of the present invention was observed.

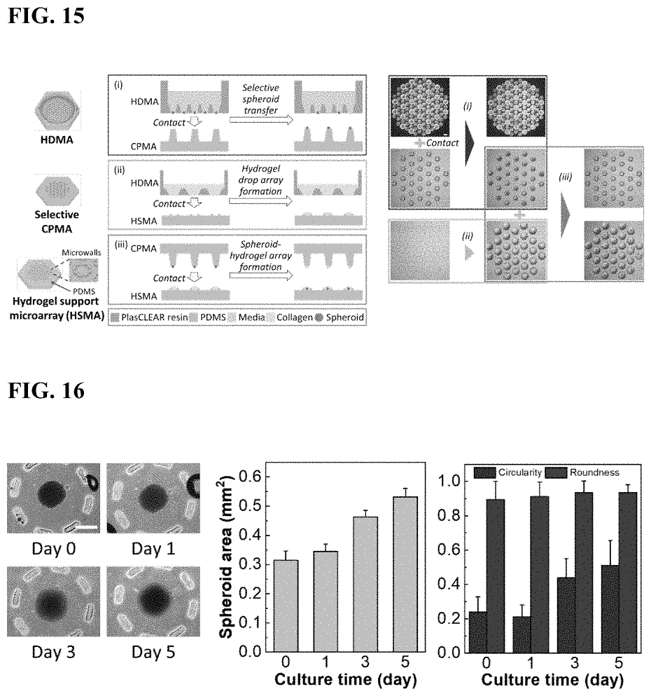

First, a portion of selectively generated spheroids was trapped using HDMA and a concave pillar microarray (CPMA) to prepare an isolated spheroid droplet array (FIG. 15, in part (i) therein).

The droplet trapping array chip having microwalls including air channels on the plane prepared in Example 8-2 was brought into contact with HDMA loaded with collagen hydrogel without spheroids for secondary transfer from the CPMA to transfer only the collagen hydrogel thereby form droplets, thereby producing a hydrogel support microarray (HSMA) (FIG. 15, in part (ii) therein).

The CPMA was flipped and the spheroid droplets trapped on the CPMA was brought into contact with the collagen hydrogel droplets on the HSMA to transfer the spheroids to the HSMA, thereby forming a spheroid-hydrogel droplet array (FIG. 15, in part (iii) therein).

In the finally produced spheroid-hydrogel droplet array, whether or not the spheroids were transferred without damage was determined. As shown in FIG. 16, the result showed that no damage to the spheroids occurred during the transfer to the hydrogel droplets, and that the size, circularity, and roundness all increased over time as they were stably cultured in the hydrogel droplets.

The droplet trapping structure array according to the present invention and the method/device for transferring spheroids using the same advantageously enable droplets or spheroids to be transferred with very high efficiency and have very small variation between users by simply contacting the two arrays.

The spheroid transfer method and device of the present invention enable mass-production of spheroid arrays in an isolated environment. In particular, the droplet trapping structure array and the spheroid transfer method of the present invention can be useful for treatment of spheroids with various reagents and exchange of culture media.

### INDUSTRIAL APPLICABILITY

The droplet-trapping structure array and the method and device for transferring spheroids using the same according to the present invention have the advantages of transferring droplets or spheroids with very high efficiency and very small variation between users by simply contacting two arrays.

The spheroid transfer method and device according to the present invention enable mass-production of spheroid arrays in an isolated environment. In particular, the droplet trapping structure array and the spheroid transfer method of the present invention can be useful for treatment of spheroids with various reagents and exchange of culture media.

Although specific configurations of the present invention have been described in detail, those skilled in the art will appreciate that this detailed description is provided as preferred embodiments for illustrative purposes and should not be construed as limiting the scope of the present invention. Therefore, the substantial scope of the present invention is defined by the accompanying filed claims and equivalents thereto.

1. A contact-based droplet trapping structure array comprising a concave plateau,
wherein the concave plateau is
(i) formed with at least one microwall; or
(ii) formed on a pillar top surface.

2. The contact-based droplet trapping structure array according to claim 1, wherein the concave plateau has a hemispherical or semi-elliptical shape.

3. The contact-based droplet trapping structure array according to claim 1, wherein the concave plateau comprises an air channel.

4. The contact-based droplet trapping structure array according to claim 1, wherein the droplet comprises a spheroid.

5. The contact-based droplet trapping structure array according to claim 4, wherein a width of the concave plateau is larger than that of the spheroid.

6. The contact-based droplet trapping structure array according to claim 4, wherein the width of the concave plateau is at least 300 μm larger than that of the spheroid.

7. A spheroid transfer device comprising:
a spheroid array; and
the contact-based droplet trapping structure array according to claim 1.

8. The spheroid transfer device according to claim 7, wherein the spheroid array is a hanging drop array.

9. The spheroid transfer device according to claim 7, wherein the hanging drop array comprises a plurality of holes at a bottom thereof and droplets containing spheroids are formed in the holes.

10. The spheroid transfer device according to claim 7, further comprising a spacer.

11. The spheroid transfer device according to claim 7, wherein when the spheroid array is brought into contact with a concave plateau of the contact-based droplet trapping structure array, the droplets containing spheroids are trapped in the concave plateau.

12. The spheroid transfer device according to claim 7, wherein the spheroid array is brought into contact with the concave plateau on a top of the contact-based droplet trapping structure array.

13. A spheroid transfer method comprising:
(a) bringing a spheroid array into contact with the contact-based droplet trapping structure array according to claim 1; and
(b) releasing the contact between the spheroid array and the contact-based droplet trapping structure array to transfer droplets containing spheroids to the concave plateau of the contact-based droplet trapping structure array.

14. The method according to claim 13, wherein step (a) comprises bringing the spheroid array into contact with a top of the concave plateau of the contact-based droplet trapping structure array.

15. A method of treating spheroids with a reagent, comprising:
(a) bringing a cultured spheroid array into contact with the contact-based droplet trapping structure array according to claim 1;
(b) releasing the contact between the cultured spheroid array and the contact-based droplet trapping structure array to produce an isolated spheroid droplet array; and
(c) bringing the isolated spheroid droplet array into contact with a reagent-loaded array.

16. The method according to claim 15, further comprising, before step (c), (b′) washing while bringing the isolated spheroid droplet array in step (b) into contact with a drop array loaded with washing buffer at the bottom of the drop array and then releasing the contact.

17. The method according to claim 15, wherein in step (c), the spheroids are treated with the reagent by bringing the isolated spheroid droplet array into contact with the bottom of the reagent-loaded array.

18. A method of exchanging a spheroid medium, comprising:
(a) bringing a cultured spheroid array into contact with the contact-based droplet trapping structure array according to claim 1;
(b) releasing the contact between the cultured spheroid array and the contact-based droplet trapping structure array to produce an isolated spheroid droplet array; and
(c) bringing the isolated spheroid droplet array into contact with a medium-loaded drop array to transfer spheroids.

19. The method according to claim 18, wherein in step (c), the spheroid medium is exchanged with a fresh medium by bringing the isolated spheroid droplet array into contact with a top of the medium-loaded array.
